(19) United States
(12) Patent Application Publication
(10) Pub. No.: US 2013/0197515 A1
(43) Pub. Date: Aug. 1, 2013

(54) ENDOSCOPIC INSTRUMENT HAVING A CUTTING TOOL

(71) Applicants: Samuel Raybin, Marlborough, MA (US); Naroun Suon, Lawrence, MA (US); Paul Smith, Smithfield, RI (US); Ruth Cheng, Natick, MA (US); Oscar Carrillo, Jr., Attleboro, MA (US)

(72) Inventors: Samuel Raybin, Marlborough, MA (US); Naroun Suon, Lawrence, MA (US); Paul Smith, Smithfield, RI (US); Ruth Cheng, Natick, MA (US); Oscar Carrillo, Jr., Attleboro, MA (US)

(73) Assignee: Boston Scientific Scimed, Inc., Maple Grove, MN (US)

(21) Appl. No.: 13/753,837

(22) Filed: Jan. 30, 2013

Related U.S. Application Data
(60) Provisional application No. 61/592,763, filed on Jan. 31, 2012.

(51) Int. Cl. A61B 17/32; A61B 18/14

(52) U.S. Cl. CPC A61B 17/320016; A61B 18/14
USPC 606/46; 606/170

(57) ABSTRACT

An embodiment of the invention is directed to a medical instrument that includes a flexible elongate member and a distal assembly. The distal assembly is coupled to a distal end of the elongate member. The distal assembly includes a blunt member and a cutting tool pivotably attached to the blunt member. The blunt member is configured for insertion into a tissue layer. The cutting tool is arranged relative to the blunt member so that the cutting tool does not cut tissue as the blunt member is inserted into the tissue layer.

### RELATED APPLICATION(S)

This application claims the benefit of priority of U.S. Provisional Application No. 61/592,763 filed Jan. 31, 2012, which is incorporated herein by reference in its entirety.

### DESCRIPTION OF THE INVENTION

1. Field of the Invention

Embodiments of the present invention relate generally to medical instruments. More particularly, embodiments of the invention relate to a medical instrument having a cutting tool for use in medical applications, such as, for example, dissection procedures. Embodiments of the disclosure also cover methods of using such instruments.

2. Background of the Invention

Endoscopes may be used in the diagnosis and/or treatment of a wide range of diseases and disorders that typically require a physician to access and navigate internal anatomical lumens within a patient's body. In use, an endoscope may be positioned in a desired body portion, and a treatment instrument may be advanced through a working channel of the endoscope to the desired body portion.

For example, in certain tissue dissection procedures, a cutting tool, such as a surgical blade, may be directed through the working channel of an endoscope, and the endoscope may be maneuvered to a desired tissue site within the body portion. At the tissue site, the physician may insert the cutting tool to excise the desired tissue portion.

During some dissection procedures, it may be required to dissect a single tissue layer while leaving the underlying tissue layer intact. Difficulties with visualization may make standard cutting tools dangerous during such procedures. Specifically, physicians may be unable distinguish between different types of tissue at the tissue site. Moreover, the standard cutting tools may include one or more sharp edges or points, which may inadvertently damage the underlying tissue layer during the course of a dissection procedure. With some tools, physicians may be unable to control depth of the cutting tool as it is extended into the tissue site, risking perforation of the underlying tissue. As such, there exists a need for a safe cutting tool that addresses one or more of these concerns.

### SUMMARY OF THE INVENTION

Embodiments of the present invention are directed to medical instruments with a cutting tool and methods of use that obviate one or more of the limitations and disadvantages of the known tools.

One embodiment of the invention is directed to a medical instrument. The medical instrument may include an elongate member and a distal assembly coupled to a distal end of the elongate member. The distal assembly may include a blunt member and a cutting tool pivotably attached to the blunt member. The blunt member may be configured for insertion into a tissue layer, and the cutting tool may be arranged relative to the blunt member so that the cutting tool does not cut tissue as the blunt member is inserted into the tissue layer.

In various embodiments, the medical instrument may include one or more of the following additional features: wherein the distal assembly further includes a clevis; wherein the blunt member is integral with the clevis; wherein the cutting tool is disposed within the clevis; wherein the cutting tool includes a raised cutting portion; wherein the cutting tool is configured to conduct energy; wherein the cutting tool is configured to rotate relative to the blunt member; wherein the cutting tool is configured to be received in a cavity of the blunt member when the blunt member is inserted in the tissue layer; wherein the cutting tool is configured to cut the tissue layer above the blunt member; and wherein the blunt member is configured to minimize damage to tissue below the blunt member.

Another embodiment of the invention is directed to a medical instrument. The medical instrument may include an elongate member having a distal end and a distal assembly extending distally of the distal end of the elongate member. The distal assembly may include a blunt member and a cutting tool pivotably attached to the blunt member. The cutting tool may be configured to dissect a tissue layer and the blunt member may be configured to reduce damage to adjacent tissue during the dissection procedure.

In various embodiments, the medical instrument may include one or more of the following additional features: wherein the distal assembly further includes a clevis; wherein the blunt member is integral with the clevis; wherein the cutting tool is configured to move relative to the blunt member between a first position and a second position, and wherein the blunt member is disposed between the cutting tool and adjacent tissue; wherein the cutting tool contacts the blunt member in the first position; wherein the blunt member is insulated from the cutting tool; wherein the cutting tool is rotated away from the blunt member and adjacent tissue in the second position; wherein the cutting tool includes a cutting portion; wherein the cutting portion is configured to dissect tissue upon being energized by an electrical current; and wherein the blunt member is configured to control rotation of the cutting tool.

Another embodiment is directed to a method of performing a dissection procedure. The method may include positioning a distal portion of a medical instrument adjacent a tissue site. The distal portion of the medical instrument may include an elongate member and a distal assembly coupled to a distal end of the elongate member. The distal assembly may include a blunt member and a cutting tool pivotably attached to the blunt member. The method may also include inserting the blunt member into tissue. In addition, the method may include dissecting the tissue and limiting contact between the cutting tool and tissue adjacent to the blunt member.

In various embodiments, the method may include one or more of the following additional features: further comprising rotating the cutting tool relative to the blunt member between a first position and a second position, and wherein the blunt member is disposed between the cutting tool and adjacent tissue; wherein the cutting tool substantially contacts the blunt member in the first position; wherein the cutting tool is rotated away from the blunt member; wherein dissecting the tissue includes dissecting the tissue when the cutting tool moves between the first position and the second position; wherein the cutting portion conducts energy.

It is to be understood that both the foregoing general description and the following detailed description are exemplary and explanatory only and are not restrictive of the invention, as claimed.

### BRIEF DESCRIPTION OF THE DRAWINGS

FIG. 1 illustrates an endoscope for use with a medical instrument having a distal assembly including a blunt member and a cutting tool, according to an embodiment of the invention;

FIG. 2 is a partial perspective view of a distal portion of the medical instrument with the cutting tool in a first position relative to the blunt member, according to a first embodiment of the invention;

FIG. 3 is a partial perspective view of the distal portion of the medical instrument with the cutting tool in a second position relative to the blunt member, according to a first embodiment of the invention;

FIG. 4 is a partial perspective side view of the distal portion of the medical instrument of FIG. 3 with a portion of the distal portion (i.e., a portion of an elongate member, a clevis, and the blunt member) removed, according to a first embodiment of the invention;

FIG. 5 is a top view of the blunt member integrally formed with the clevis, according to a first embodiment of the invention;

FIG. 6 is a perspective side view of the cutting tool, according to a first embodiment of the invention;

FIG. 7A illustrates the distal assembly of the medical instrument being positioned at a tissue site, according to a first embodiment of the invention;

FIG. 7B illustrates the distal assembly being inserted between two adjacent tissue layers with the cutting tool in the first position, according to a first embodiment of the invention;

FIG. 7C illustrates movement of the cutting tool from the first position to the second position to dissect a tissue layer, according to a first embodiment of the invention;

FIG. 8 is a partial perspective side view of a distal portion of the medical instrument including a distal assembly with a blunt member and a cutting tool, the cutting tool is in a first position relative to the blunt member, according to a second embodiment of the invention;

FIG. 9 is a partial perspective side view of a distal portion of the medical instrument with the cutting tool in a second position relative to the blunt member, according to a second embodiment of the invention;

FIG. 10A illustrates the distal assembly of the medical instrument being positioned at a tissue site, according to a second embodiment of the invention;

FIG. 10B illustrates a blunt member of the distal assembly being inserted between two tissue layers with the cutting tool in the second position, according to a first embodiment of the invention; and

FIG. 10C illustrates movement of the cutting tool from the second position to the first position to dissect a tissue layer, according to a second embodiment of the invention.

### DESCRIPTION OF THE EMBODIMENTS

Reference will now be made in detail to exemplary embodiments of the invention, examples of which are illustrated in the accompanying drawings. Whenever possible, the same reference numbers will be used throughout the drawings to refer to same or like parts.

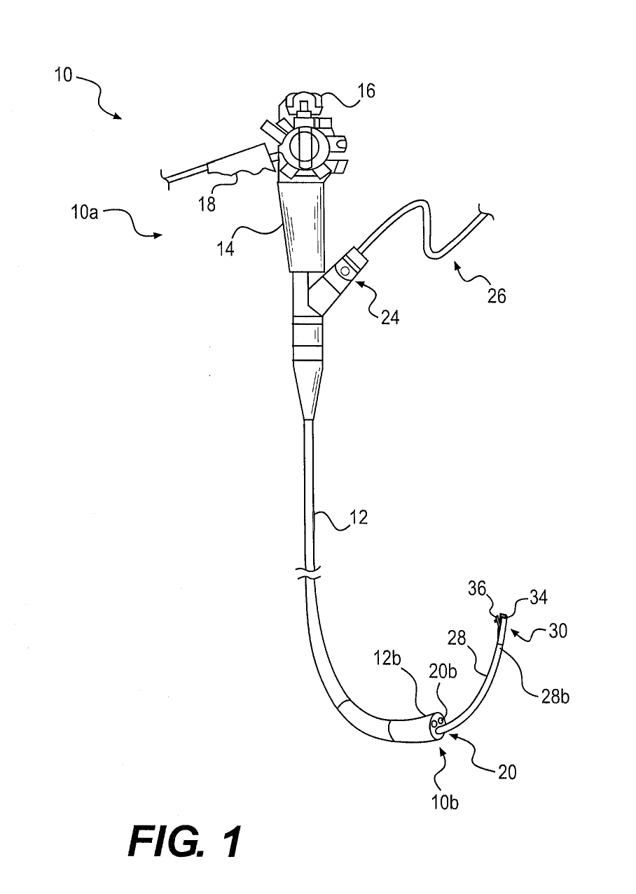

FIG. 1 depicts an endoscope 10 according to an exemplary embodiment of the invention. Endoscope 10 may be used for procedures within or adjacent to various body organs, such as, an esophagus, a heart, a stomach, a pelvic area, a bladder, an intestine, or any other portion of the body including a gastrointestinal, urinary, or pulmonary tract.

Endoscope 10 may be configured for insertion into a patient's body through an anatomical opening. In some embodiments, endoscope 10 may be used in natural orifice transluminal endoscopic surgery (NOTES) procedures or single incision laparoscopic surgical (SILS) procedures or other percutaneous procedures. Accordingly, endoscope 10 may be shaped and sized for placement into a patient via a body cavity or an incision.

Endoscope 10 may include a proximal end 10a, a distal end 10b, and an outer tube 12 extending between proximal end 10a and distal end 10b. For purposes of this disclosure, “proximal” refers to the end closer to the device operator during use, and “distal” refers to the end further from the device operator during use.

A handle portion 14 may be disposed at proximal end 10a of endoscope 10. Handle portion 14 may be any known, suitable handle. As illustrated in FIG. 1, handle portion 14 may include rotatable control knobs 16, which may be connected to control wires or cables (not shown) within outer tube 12, to provide up/down and left/right steering of distal end 10b of endoscope 10. Handle portion 14 may additionally include an adapter 18 for allowing delivery of energy such as electrical and/or radiofrequency energy to distal end 10b of endoscope 10.

Outer tube 12 may extend distally from handle portion 14 and may terminate at a distal end 12b. Outer tube 12 may be a flexible tube, made from any suitable biocompatible material known to one of ordinary skill in the art and having sufficient flexibility to traverse tortuous anatomy. Such materials may include, but are not limited to, rubber, silicon, polymers, stainless steel, metal-polymer composites, and metal alloys of nickel, titanium, copper cobalt, vanadium, chromium, and iron. In one embodiment, the material forming outer tube 12 may be a superelastic material such as nitinol, which is a nickel-titanium alloy. In some embodiments, outer tube 12 may include layers of different materials and reinforcements such as braiding or coiling within the wall of outer tube 12. Outer tube 12 may have any cross-sectional shape and/or configuration and may be any desired dimension that can be received in a body cavity. In some embodiments, outer tube 12 may be made of, or coated with, a polymeric or lubricious material to enable endoscope 10 to pass through a body cavity with ease. Additionally, outer tube 12 may be steerable and may have areas of different flexibility or stiffness to promote steerability with the body cavity. Steerability may, for example, be controlled by wires.

Outer tube 12 may include one or more channels 20. The one or more channels 20 may extend substantially longitudinally (axially) within outer tube 12, and generally between proximal end 10a and distal end 10b of endoscope 10. In particular, the one or more channels 20 may extend distally from handle portion 14 and terminate at an aperture 20b at distal end 12b of outer tube 12. The one or more channels 20 may have any suitable size, cross-sectional area, shape, and/or configuration to, for example, introduce medical instruments to distal end 10b of endoscope 10.

In the exemplary embodiment depicted in FIG. 1, outer tube 12 includes three channels 20. A medical instrument 26 may be introduced through one of the three channels. The additional channels may introduce visualization devices (i.e., lighting sources and/or imaging sources) to distal end 10b of endoscope 10. It should be understood, however, that outer tube 12 may include a greater or lesser number of channels. It is contemplated that additional channels may be provided for irrigation and/or aspiration.

Medical instrument 26 may be slidably inserted through a port 24 at proximal end 10a of endoscope 10 to enter channel 20. As shown in FIG. 1, port 24 may be provided at an angle to channel 20 in outer tube 12. Medical instrument 26 may be advanced through channel 20, and a distal portion 26b of medical instrument 26 may be positioned distally of aperture 20b. Distal portion 26b of medical instrument 26 may include distal assembly 30 attached to a distal end 28b of an elongated member 28. Distal assembly 30 may be configured for use during a surgical method including diagnostic and/or therapeutic procedures. Specifically, distal assembly 30 may be configured for use in dissection procedures such as, for example, an endoscopic submucosal dissection (ESD) procedure.

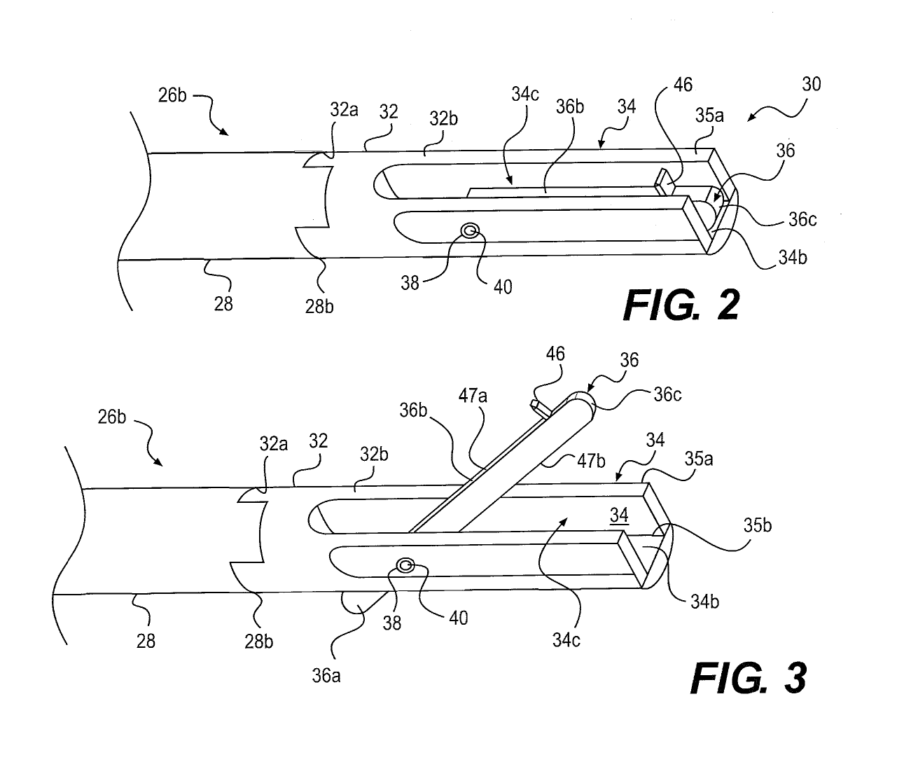

FIGS. 2-6 depict an exemplary distal assembly 30 and the components thereof in accordance with a first embodiment of the invention. Distal assembly 30 may include a clevis 32, a blunt member 34 integrally formed with clevis 32, and a cutting tool 36 pivotably attached to blunt member 34. Cutting tool 36 may include a proximal portion 36a, a central portion 36b, a distal portion 36c, and a cutting portion 46 mounted on a top surface 47a of distal portion 36c.

As will be described in more detail below, cutting tool 36 may be configured to move relative to blunt member 34 between a first position shown in FIG. 2 and a second position shown in FIG. 3. In the first position, cutting tool 36 may be received in blunt member 34 so that at least cutting portion 46 does not extend beyond the tops of the arms of blunt member 34. In the second position, cutting tool 36 may be deflected away from blunt member 34. Blunt member 34 may be configured to prevent cutting tool 36 from rotating past the center axis of distal assembly 30. As a result, cutting portion 46 of cutting tool 36 may create a cutting action against a targeted tissue layer in only one direction relative to the central axis of blunt member 34 when cutting tool 36 moves from the first position to the second position, and blunt member 34 may limit perforation and/or damage of an adjacent tissue layer.

Referring to FIGS. 2-5, clevis 32 of distal assembly 30 may have a proximal end 32a, a pair of arms 32b, and a throughhole 32c extending from proximal end 32a to a space formed between arms 32b. Proximal end 32a may be affixed to distal end 28b of elongate member 28 using any conventional means, such as, for example, crimping, swagging, gluing, laser welding, or any other method of adhering the clevis 32 to elongate member 28. Arms 32b may extend distally from proximal end 32a of clevis 32. Arms 32b may be substantially similar in shape, however, they may also have different shapes or configurations. In some embodiments, the edges and corners of the arms 32b of clevis 32 may be rounded to avoid damage to tissue and channel 20 of endoscope 10.

Blunt member 34 may be fixed to clevis 32 and, thus, may be immovable relative to clevis 32 and elongate member 28. In accordance with the first exemplary embodiment, blunt member 34 may be a jaw having a pair of side walls 34a, a base wall 34b, and a central cavity 34c. Side walls 34a may be vertical inner walls of arms 32b of clevis 32. In particular, blunt member 34 and clevis 32 may be formed as a unitary mold or cast member from a biocompatible metal, polymer, or any other material having sufficient strength (e.g., plastic ceramic composite). Each side wall 34a may have an axle hole 38 for receiving an axle pin 40 (FIGS. 2 and 3). Each side wall 34a of blunt member 34 may further define a top 35a and a bottom 35b. Base wall 34b may extend between bottoms 35b of side walls 34a, and transverse to side walls 34a. In the exemplary embodiment, an inner surface of base wall 34b may be substantially flat. It is contemplated, however, that inner surface of base wall 34b may have any other shape and/or configuration to receive cutting tool 36. In some embodiments, an outer surface of base wall 34b may be rounded so as to minimize damage to tissue and channel 20 of endoscope 10. It will be appreciated that blunt member 34 is not restricted in shape or cross-section or to an open distal channel. For example, side walls 34a may be angled towards each other at distal end of blunt member 34. The distal end may be open on top or may have an enclosed blunt shape (e.g., bullet, boathull, rounded, etc.).

Central cavity 34c may be formed by side walls 34a and base wall 34b. Central cavity 34c may have an open top defined by tops 35a of side walls 34a and a closed bottom (i.e., base wall 34b). Central cavity 34c may be aligned with the space between arms 32b of clevis 32. Central cavity 34c may have a cross-sectional area that is larger than the cross-sectional area of cutting tool 36, and may have substantially the same cross-sectional shape as cutting tool 36. In some embodiments, base wall 34 may include a groove to receive cutting tool 36. In the exemplary embodiment, central cavity 34c may have a substantially rectangular shape along its length, however, it is understood that central cavity 34c may have any shape and/or dimensions to receive cutting tool 36.

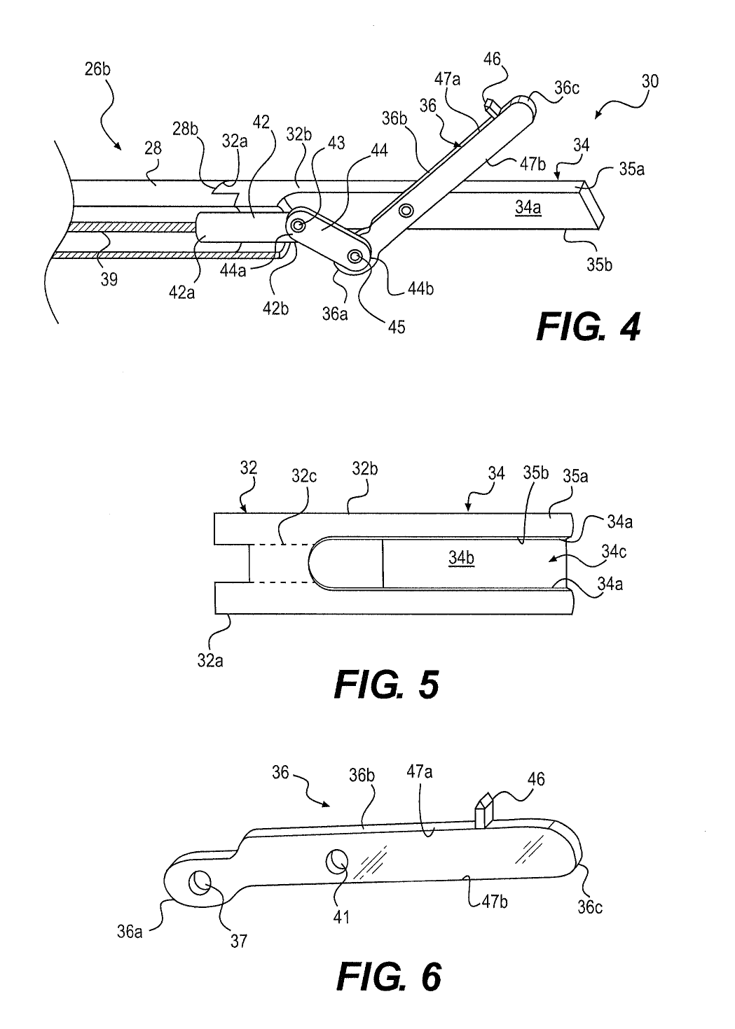

Cutting tool 36 may be disposed between arms 32b of clevis 32 and side walls 34a of blunt member 34. An actuator 42 may be received in throughhole 32c of clevis 32 and may be used to rotate cutting tool 36 relative to clevis 32 and blunt member 34. More particularly, an actuation wire 39 (FIG. 4) may be attached to a proximal end 42a of actuator 42, and extend proximally through elongate member 28 of medical instrument 26 to proximal end 10a of endoscope 10. A distal end 42b of actuator 42 may be connected to a proximal end 44a of a link 44 near the distal end 42b of actuator 42 via a first pin 43. A distal end 44b of link 44 may be connected, in turn, to a proximal portion 36a of cutting tool 36 via a second pin 45. Link 44 may receive first pin 43 and second pin 45 anywhere along the length or height of link 44 to provide the desired control to link 44 and cutting tool 36. Link 44 may have any shape and/or configuration including, but not limited to, a rectangular shape, a curved shape, an oblong shape, an elliptical shape, or a tapered shape. As illustrated in FIG. 6, proximal portion 36a of cutting tool 36 may define a mounting hole 37 to accommodate second pin 45.

An axle hole 41 may be disposed on central portion 36b of cutting tool 36, and may be configured to accommodate pivot pin 40 to pivotably attach cutting tool 36 to blunt member 34. In this configuration, forward, distal movement of actuator 42 may translate force through link 44 to rotate distal portion 36b of cutting tool 36 about pivot pin 40 from the first position shown in FIG. 2 to the second position shown in FIG. 3. Conversely the rearward, proximal movement of actuator 42, may translate force through link 44 to pivot cutting tool 36 about pivot pin 40 from the second position shown in FIG. 3 to the first position shown in FIG. 2. Distal portion 36c of cutting tool 36 may be configured to be received in central cavity 34c when cutting tool 36 is in the first position shown in FIG. 2, and distal portion 36c of cutting tool 36 may be rotated away from the open top of central cavity (i.e., tops 35a of side walls 34a) when cutting tool 36 is in the second position shown in FIG. 3. It is contemplated that in alternative embodiments, blunt tool 34 may pivot inside and relative to a fixed cutting tool 36.

In accordance with the first embodiment, distal portion 36c of cutting tool 36, shown in FIG. 6, may be a jaw having, with the central portion 36b, a bottom surface 47b and top surface 47a. Central and distal portions 36b,c may be configured so as to minimize damage and/or perforation to tissue. In the exemplary embodiment, central and distal portions 36b,c may rounded at least near the edges so that there are no, or fewer, sharp edges or corners associated with central and distal portions 36b,c of cutting tool 36.

Bottom surface 47b of central and distal portions 36b,c of cutting tool 36 may be configured to contact base wall 34b of blunt member 34 when cutting tool 36 is disposed in the first position. In the exemplary embodiment, bottom surface 47b may be substantially flat such that bottom surface 47b may contact and rest on base wall 34b. Top surface 47a may be may be tapered or sloped downward to create a wedge effect with blunt member 34. Top surface 47 may have any other shape and/or configuration.

Cutting portion 46 may be mounted on top surface 47a of distal portion 36c of cutting tool 36. In the exemplary embodiment, cutting portion 46 is welded or otherwise adhered to top surface 47a of distal portion 36b of cutting tool 36. It is also contemplated that cutting portion 46 may be received in a groove formed on top surface 47a, or may be integrally formed with cutting tool 36 by welding, molding, or stamping methods.

Cutting portion 46 may be configured to cut tissue. In the preferred embodiment, cutting portion 46 may be a double-edged blade. Alternatively, cutting portion 46 may be a serrated blade, a curved blade having a dull edge, or any other known blade type. Either edge of cutting portion 46 may have features the same or different to the features on the other edge. In some embodiments, cutting portion 46 may be any other known dissection tool including, but not limited to, a needle, a knife, a pin, or a hook. In embodiments including a pin, the pin may be arranged substantially perpendicular to top surface 47a. In embodiments include a hook, the hook may be arranged in parallel to top surface 47a, facing proximally and or distally. In other embodiments, cutting portion 46 may be a raised curvilinear section of top surface 47a of cutting tool 36.

Cutting portion 46 may be electrically conductive. For example, cutting portion 46 may dissect tissue upon being energized by an electrical current. In one exemplary embodiment, wire 39, actuator 42, and link 44, may provide an electrical pathway from a source of electrical current to cutting portion 46. Wire 39, actuator 42, and link 44 may be formed of any material capable of conducting electricity, such as, for example, stainless steel, nickel titanium alloys, and the like. Cutting tool 36 may also be formed of any material capable of conducting electricity; however top surface 47a and bottom surface 47b of cutting tool 36 may be covered with a suitable insulating material, such as, for example, a powder or liquid coat or non-conducting polymeric sheath, to minimize the discharge and effects of any stray electrical energy from cutting portion 46. Insulation of cutting tool 36 may also prevent electrical energy from causing tissue damage due to incidental contact with cutting tool 36. Similarly, blunt member 34, clevis 32, and elongate member 28 may be formed of any insulating material such as non-conducting polymer material, or may be coated with an insulating material incapable of conducting electricity such as a polymer or ceramic material.

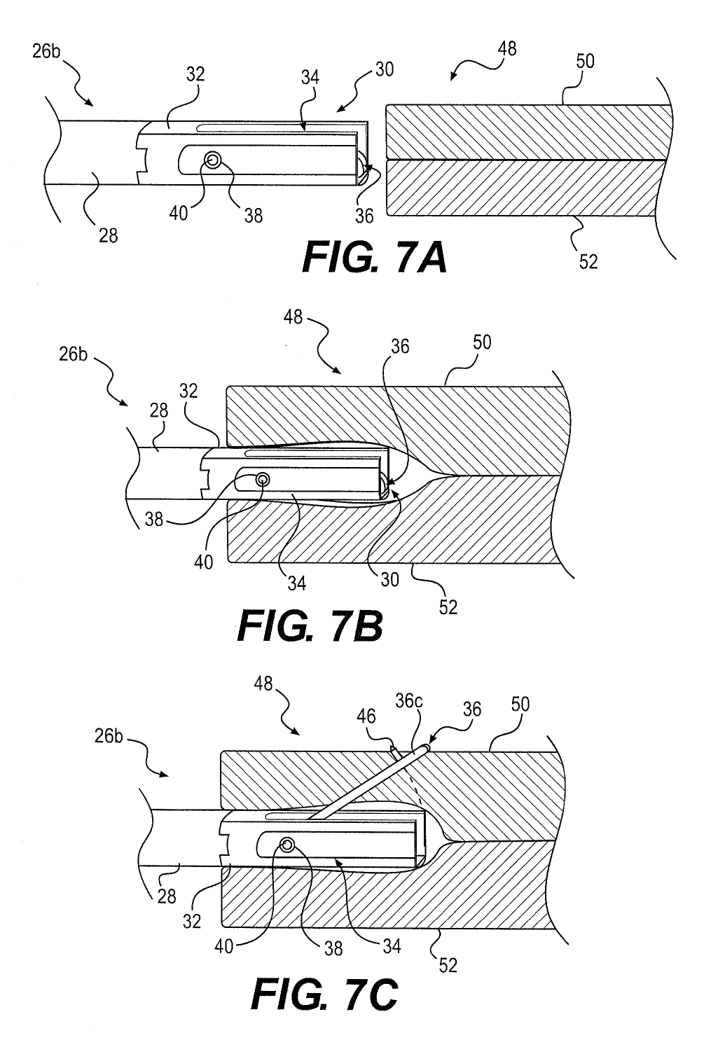

A method of using medical instrument 26 will now be described. Once an endoscope 10 is provided at the site of the procedure, distal portion 26b of medical instrument 26 may be advanced through channel 20 of endoscope 10 to distal end 10b of endoscope 10. Distal portion 26b may be maneuvered to a tissue site 48 so that distal assembly 30 is positioned adjacent tissue site 48 (FIG. 7A).

As will be discussed in more detail below, the disclosed distal assembly 30 may include a safe cutting tool for use during a dissection procedure. In particular, blunt member 24 of distal assembly 30 may be configured to minimize perforation and/or damage of tissue by cutting portion 46 of cutting tool 36 when cutting tool 36 is inserted into tissue site 48. Moreover, during the dissection procedure, the disclosed distal assembly 30 may enable a physician to dissect a desired tissue layer while leaving an adjacent tissue layer intact.

Referring to FIG. 7B, distal assembly 30 may be oriented so that, upon insertion of distal assembly 30 into tissue site 48, cutting tool 36 may be positioned beneath the tissue layer to be dissected and blunt member 34 may be positioned between cutting tool 36 and the tissue layer to be protected. In one embodiment, cutting tool 36 may be inserted between the muscularis and mucosal layers. In other embodiments, cutting tool 36 may be inserted into a soft tissue layer like the submucosa and oriented so that the tool pivots in the plane of the tissue layer.

Prior to insertion, cutting tool 36 may be rotated relative to blunt member 34 so that cutting tool 36 is in a first position. More particularly, cutting tool 36 may be rotated relative to blunt member 34 so that cutting tool 36, including cutting portion 46, may be positioned within central cavity 34c of blunt member 34. Blunt member 34 and cutting tool 36 may then be safely inserted into a tissue site 48 without perforating and/or damaging either the targeted tissue layer 50 or an adjacent tissue layer 52 (FIG. 7B).

After blunt member 34 and cutting tool 36 have been inserted between tissue layers 50, 52, cutting tool 36 may be rotated relative to blunt member 36 from the first position (FIG. 7B) to the second position (FIG. 7C). In the second position, the cutting tool 36 may be deflected away from blunt member 34. As blunt member 34 may prevent cutting tool 36 from rotating past the center axis of distal assembly 30, cutting tool 36 may move in only one direction relative to a central axis of blunt member 34. In doing so, cutting portion 46 may cut against the targeted tissue layer 50. During this step, cutting portion 46 may be energized by an electrical current so that cutting portion 46 may dissect the targeted tissue layer 50.

As illustrated in FIGS. 7B and 7C, blunt member 34 may be positioned between cutting tool 36 and the adjacent tissue layer 52 when cutting tool 36 is in the first position and the second position. In this manner, blunt member 34 may minimize damage and/or perforation to the adjacent tissue layer 52 during the dissection procedure.

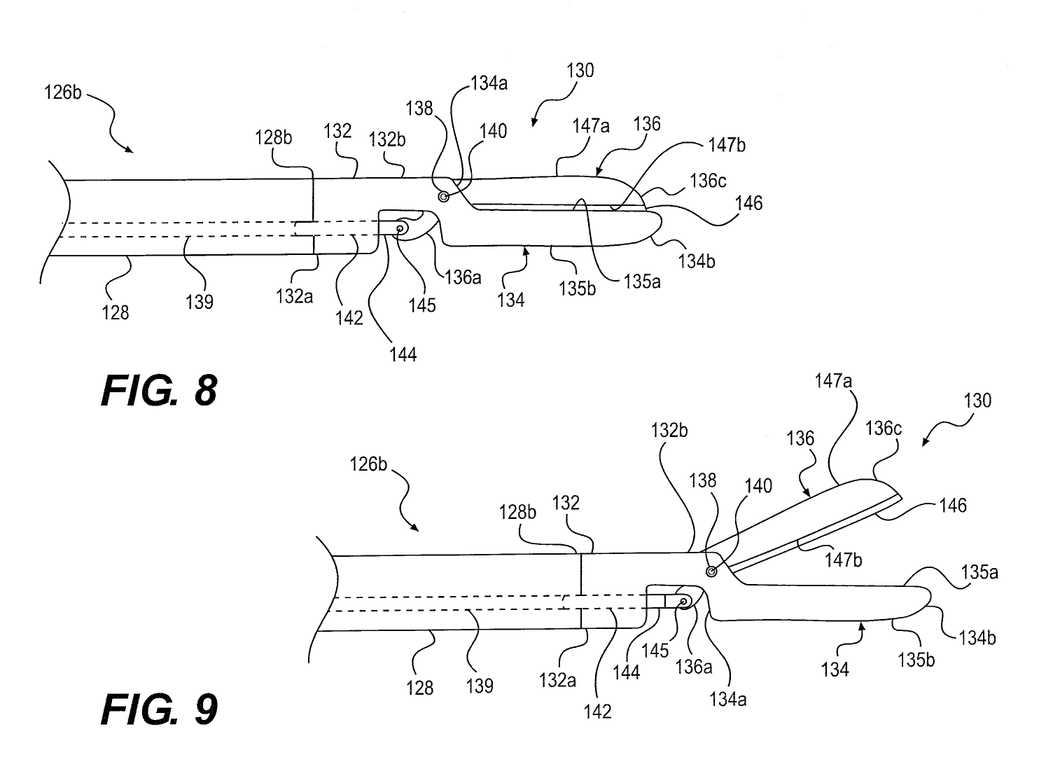

FIGS. 8 and 9 depict an exemplary distal assembly 130 and the components thereof in accordance with a second embodiment of the invention. Distal assembly 130 may include an assembly of multiple components including a clevis 132, a blunt member 134 integrally formed with clevis 132, and a cutting tool 136 pivotably attached to blunt member 134. In accordance with the second embodiment, cutting tool 136 may include a cutting portion 146 disposed along a bottom surface 147b of a distal portion 136c of cutting tool 136.

Distal assembly 130 may be similar to distal assembly 30 of the first embodiment. In particular, cutting tool 136 may be configured to move relative to blunt member 134 between a first position shown in FIG. 8 and a second position shown in FIG. 9. In the second embodiment, however, cutting portion 146 of cutting tool 136 may be configured to cut tissue as cutting tool 136 moves from the second position to the first position.

As in the first embodiment, blunt member 134 may be configured to prevent cutting tool 136 from rotating past a central longitudinal axis of distal assembly 130. Accordingly, cutting portion 146 of cutting tool 136 may dissect the targeted tissue layer, and blunt member 134 may limit perforation and/or damage of an adjacent tissue layer during the dissection procedure.

Referring to FIGS. 8 and 9, clevis 132 of distal assembly 130 may have a proximal end 132a and a pair of arms 132b. Proximal end 132a may be affixed to distal end 128b of elongate member 128 using any conventional means, such as, for example, crimping, swagging, gluing, laser welding, or any other method of adhering the clevis 132 to elongate member 128. Arms 132b may extend distally from proximal end 132a of clevis 132. Arms 132b may be substantially similar in shape, however, they may also have different shapes or configurations.

Blunt member 134 may be fixed to clevis 132. in other words, blunt member 134 may be immovable relative to clevis 132 and elongate member 128. In accordance with the second embodiment, blunt member 134 may be a probe having a bridging portion 134a and a distal portion 134b. Bridging portion 134a may include vertical walls that are integral with arms 132b of clevis 132. In particular, blunt member 134 and clevis 132 may be formed as a unitary mold or cast member from a biocompatible metal or any other material having sufficient strength (e.g., plastic ceramic composite). Walls of bridging portion 134a may have an L-shape configuration defining an axle hole 138 for receiving an axle pin 140.

Distal portion 134b of blunt member 134 may be an elongate body having a top surface 135a and a bottom surface 135b. Cutting tool 136 may be configured to contact top surface 135a of distal portion 134b of blunt member 134 to limit movement of cutting tool 136 when it rotates relative to blunt member 134 between the second position and the first position. Additionally, cutting tool 136 may rest on top surface 135a of distal portion 134b of blunt member 134 when cutting tool 136 is in the first position. In the exemplary embodiment, top surface 135a may be substantially flat. It is contemplated, however, that top surface 135a of distal portion 134a of blunt member 134 may be concave or have any other shape and/or configuration. It is further contemplated that top surface 135a may be longer than cutting tool 136, and may be raised distal to cutting tool 136 to aid in dissection. In these embodiments, top surface 135a may have a groove to receive a portion of cutting tool 136.

In some embodiments, bottom portion 135b of blunt member 134 may be configured so as to minimize damage and/or perforation of tissue. In particular, the corners and edges of various intersecting surfaces on bottom surface 135b may be beveled, rounded, wedge shaped, and/or radiused off and not sharp. In the exemplary embodiment, bottom surface 135b and certain surfaces intersecting with bottom surface 135b are rounded at least near the edges so that there are no, or fewer, sharp edges or corners associated with blunt member 134.

A proximal portion 136a of cutting tool 136 may be disposed between arms 132b of clevis 132 and walls of bridging portion 134a of blunt member 134, and mounted to walls of bridging portion 134a by axle pin 140. An actuation mechanism including an actuation wire 139, an actuator 142, and a link 144 similar to an actuation mechanism described in the first embodiment may be used to actuate cutting tool 136. More particularly, an actuation wire 139 may be attached to actuator 142 and extend through elongate member 128 of medical instrument 126 to proximal end 10a of endoscope 10. Actuator 142 may be connected to link 144 which may be connected, in turn, to a proximal portion 136a of cutting tool 136 via a pin 145. Forward, distal movement of actuator 142 may translate force through link 144 to rotate distal portion 136c of cutting tool 136 about pivot pin 140 from the first position shown in FIG. 8 to the second position shown in FIG. 9. Conversely the rearward, proximal movement of actuator 142, may translate force through link 144 to pivot distal portion 136c of cutting tool 136 about pivot pin 140 from the second position shown in FIG. 9 to the first position shown in FIG. 8.

Distal portion 136c of cutting tool 136 may define a bottom surface 147b and a top surface 147a. Cutting portion 146 may be mounted on bottom surface 147b of cutting tool 136. In the preferred embodiment, cutting portion 146 may be a blade having a dull edge. It is contemplated that cutting portion 146 may be a serrated blade, a double-edged blade, or any other known blade type, including a blade with a sharp edge.

Cutting portion 146 may be electrically conductive. For example, cutting portion 146 may dissect tissue upon being energized by an electrical current. In one exemplary embodiment, wire 139, actuator 142, and link 144, may provide an electrical pathway from a source of electrical current to cutting portion 146. Wire 139, actuator 142, link 144, and cutting portion 146 may be formed of any material capable of conducting electricity, such as, for example, stainless steel, nickel titanium alloys, and the like. In other embodiments, the device may include a separate conductor extending from a proximal portion to distal portion 26b of the medical instrument 26. Cutting tool 136 may also be formed of any material capable of conducting electricity; however top surface 147a of cutting tool 136 may be covered with a suitable insulating material, such as, for example, a powder coat or non-conducting polymeric sheath, to minimize the discharge and effects of any stray electrical energy from cutting portion 146. Insulation of cutting tool 136 may also prevent electrical energy from causing tissue damage due to incidental contact with cutting tool 136. Similarly, clevis 132 and elongate member 128 may be formed of any non-conducting polymer material, or may be coated with an insulating polymer material incapable of conducting electricity.

In another exemplary embodiment, top surface 135a of distal portion 134b of blunt member 134 may include a conductive strip. In this embodiment, cutting portion 146 of cutting tool 136 may be configured to contact the conductive strip when cutting tool is in the first position. Wire 139, actuator 142, and link 144, cutting portion 146, and the conductive strip may form a bipolar circuit. It will be appreciated that blunt member 134 may alternatively include wires or other known components for conducting bipolar energy. In the exemplary embodiment, the conductive strip may be connected to a secondary wire extending proximally of distal assembly 130. The secondary wire may be formed of any material capable of conducting electricity, such as, for example, stainless steel, nickel titanium alloys, and the like. The conductive strip may also be formed of any material capable of conducting electricity; however, in this embodiment, the remainder of blunt member 134 may be covered with a suitable insulating material, such as, for example, a powder coat or non-conducting polymeric sheath, to minimize the discharge and effects of any stray electrical energy from cutting portion 146.

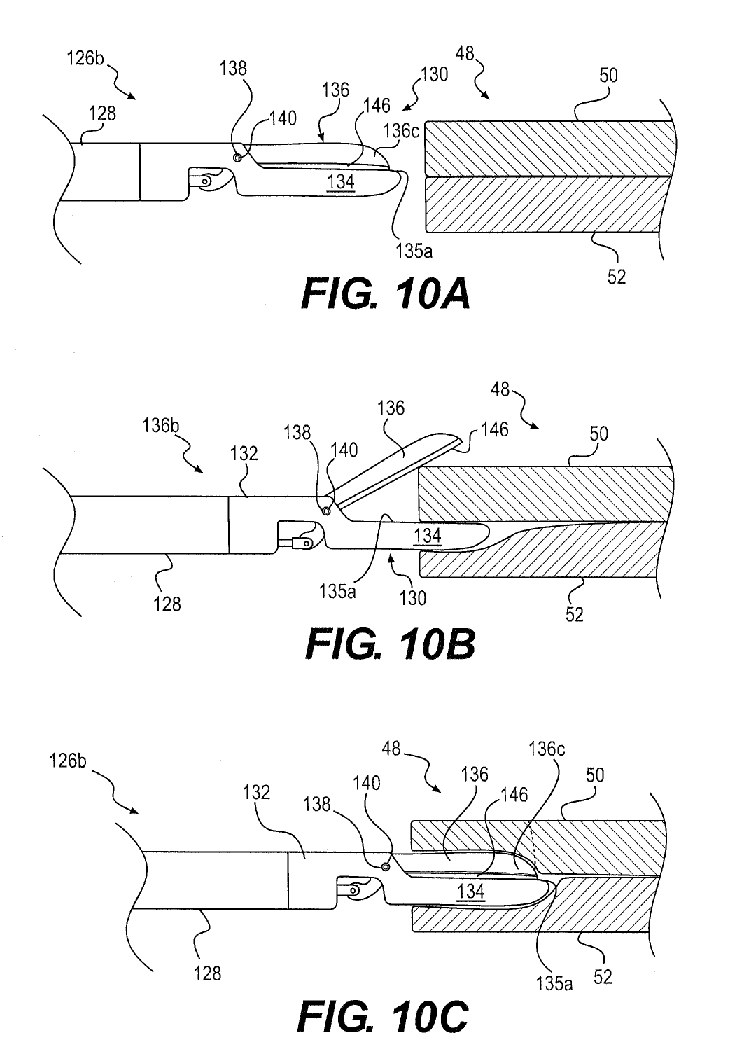

A method of using medical instrument 126 will now be described. Once an endoscope 10 is provided at the treatment site, distal portion 126b of medical instrument 126 may be advanced through channel 20 of endoscope 10 to a desired tissue site 48. Distal portion 126b may be maneuvered to tissue site 48 so that distal assembly 130 is positioned adjacent tissue site 48 (FIG. 10A).

As will be discussed in more detail below, the disclosed distal assembly 130 may include a safe cutting tool for use during a dissection procedure. In particular, blunt member 134 of distal assembly 130 may be configured to minimize perforation and/or damage of tissue by distal assembly 130 when distal assembly 130 is inserted into tissue site 48. Moreover, during the dissection procedure, the disclosed distal assembly 130 may enable a physician to dissect a desired tissue layer while leaving an adjacent tissue layer intact.

Referring to FIG. 10B, cutting tool 136 may be rotated relative to blunt member 134 so that cutting tool 136 is in a second position. More particularly, cutting tool 136 may be rotated relative to blunt member 134 so that cutting portion 146 of cutting tool 136 is deflected away from blunt member 134. Distal assembly 130 may be oriented so that blunt member 134 may be inserted between the adjacent tissue layers 50, 52.

After blunt member 134 has been inserted between the adjacent tissue layers 50, 52, cutting tool 136 may be rotated relative to blunt member 136 from the second position (FIG. 10B) to the first position (FIG. 10C). More particularly, cutting tool 136 may be rotated relative to blunt member 134 so that cutting portion 146 may cut through a tissue layer to be dissected until it contacts blunt member 134. Cutting portion 146 may be energized by an electrical current so that cutting portion 146 may dissect the targeted tissue layer 50.

During the procedure, blunt member 134 may limit movement of cutting tool 136. In particular, blunt member 134 may prevent cutting tool 136 from rotating past the center axis of distal assembly 130. In this manner, cutting portion 146 of cutting tool 136 may dissect a plane of tissue, and blunt member 134 may minimize damage to the adjacent tissue layer.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A medical instrument, comprising:
a flexible elongate member; and
a distal assembly coupled to a distal end of the elongate member, the distal assembly including a clevis, a blunt member, and a cutting tool pivotably attached to the blunt member and disposed within the clevis, wherein the blunt member is configured for insertion into a tissue layer, and wherein the cutting tool is arranged relative to the blunt member so that the cutting tool does not cut tissue as the blunt member is inserted into the tissue layer.

2. The medical instrument of claim 1, wherein the cutting tool includes a raised cutting portion.

3. The medical instrument of claim 1, wherein the cutting tool is configured to conduct energy.

4. The medical instrument of claim 1, wherein the cutting tool is configured to rotate relative to the blunt member.

5. The medical instrument of claim 1, wherein the cutting tool is configured to be received in a cavity of the blunt member when the blunt member is inserted in the tissue layer.

6. The medical instrument of claim 1, wherein the cutting tool is configured to cut the tissue layer above the blunt member.

7. The medical instrument of claim 1, wherein the blunt member is configured to minimize damage to tissue below the blunt member.

8. A medical instrument for performing a dissection procedure, comprising:
a flexible elongate member having a distal end;
a distal assembly extending distally of the distal end of the elongate member, the distal assembly including a clevis, a blunt member, and a cutting tool pivotably attached to the blunt member,
wherein the cutting tool is configured to dissect a tissue layer, and
wherein the blunt member is configured to reduce damage to adjacent tissue during the dissection procedure.

9. The medical instrument of claim 8, wherein the cutting tool is configured to move relative to the blunt member between a first position and a second position, and wherein the blunt member is disposed between the cutting tool and adjacent tissue.

10. The medical instrument of claim 9, wherein the cutting tool contacts the blunt member in the first position.

11. The medical instrument of claim 10, wherein the blunt member is insulated from the cutting tool.

12. The medical instrument of claim 9, wherein the cutting tool is rotated away from the blunt member and adjacent tissue in the second position.

13. The medical instrument of claim 8, wherein the cutting tool includes a cutting portion.

14. The medical instrument of claim 13, wherein the cutting portion is configured to dissect tissue upon being energized by an electrical current.

15. The medical instrument of claim 8, wherein the blunt member is configured to control rotation of the cutting tool.

16. A method of performing a dissection procedure, comprising:
positioning a distal portion of a medical instrument adjacent a tissue site, the distal portion of the medical instrument including an elongate member and a distal assembly coupled to a distal end of the elongate member, the distal assembly comprising a blunt member and a cutting tool pivotably attached to the blunt member;
inserting the blunt member into tissue;
dissecting the tissue; and
limiting contact between the cutting tool and tissue adjacent to the blunt member.

17. The method of claim 16, further comprising rotating the cutting tool relative to the blunt member between a first position and a second position, and wherein the blunt member is disposed between the cutting tool and adjacent tissue.

18. The method of claim 17, wherein the cutting tool substantially contacts the blunt member in the first position.

19. The method of claim 17, wherein the cutting tool is rotated away from the blunt member.

20. The method of claim 16, wherein dissecting the tissue includes dissecting the tissue when the cutting tool moves between the first position and the second position.
